(19) United States
(12) Patent Application Publication
(10) Pub. No.: US 2009/0234271 A1
(43) Pub. Date: Sep. 17, 2009

(54) COMPOSITION COMPRISING siRNA-POLYCATION COMPLEX FOR IONTOPHORESIS

(76) Inventors: Kazuaki Kajimoto, Sapporo (JP); Masahiko Yamamoto, Sapporo (JP); Kaoru Kigasawa, Sapporo (JP); Kentaro Kogure, Kyoto (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/392,883

(22) Filed: Feb. 25, 2009

Related U.S. Application Data
(60) Provisional application No. 61/088,944, filed on Aug. 14, 2008.

(30) Foreign Application Priority Data
Feb. 26, 2008 (JP) 2008044823

(51) Int. Cl. A61N 1/30; C07H 21/02; A61K 9/127; A61K 31/7088

(52) U.S. Cl. 604/20; 536/23.1; 536/24.5; 424/450; 514/44 A

(57) ABSTRACT

To provide a composition which is capable of efficiently delivering an siRNA intradermally and of effectively suppressing the expression of a target gene by an RNAi. Provided is a composition for iontophoresis including an siRNA-polycation complex which is charged negatively.

### CROSS REFERENCE TO RELATED APPLICATIONS

This application claims benefit under 35 U.S.C. 119(e) of U.S. Provisional Patent Application Ser. No. 61/088,944, filed Aug. 14, 2008, entitled “Iontophoresis Composition Containing siRNA-Polycation Compound”, which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910162_{—}529_SEQUENCE_LISTING.txt. The text file is 2 KB, was created on Feb. 25, 2009, and is being submitted electronically via EFS-Web.

### BACKGROUND

1. Technical Field

The present application relates to compositions useful in methods of intradermally administering an siRNA to an organism by iontophoresis, wherein the compositions comprise a negatively-charged siRNA-polycation complex.

2. Description of the Related Art

In recent years, an approach for alleviating or treating completely a symptom of an intractable dermatosis by specifically suppressing an expression of a disease gene intradermally as a result of local administration of a functional nucleic acid, such as a decoy or an antisense oligonucleotide, has attracted attention.

The number of patients of atopic dermatitis (AD) as a typical dermatosis has increased year by year and the prevalence in young people reaches as high as 10%. There have been studies on AD using functional nucleic acids such as a decoy or as an antisense oligonucleotide (see, for example, Nakamura, H. et al., Gene Therapy, 2002; Vol. 9, pp. 1221-1229; Yokozeki, H. et al., Gene Therapy, 2004, Vol. 11, pp. 1753-1762; Sakamoto, T. et al., Gene Therapy, 2004, Vol. 11, pp. 317-324). A therapy for AD using a functional nucleic acid is expected to be a therapy with fewer side effects than a symptomatic therapy using a steroid.

In recent years, RNA interference (RNAi) has attracted attention in the field of gene therapy. RNAi is a phenomenon of suppressing an expression of a target gene by the following method: a double-stranded RNA (dsRNA), comprising a sense strand which includes a sequence homologous to a messenger RNA (mRNA) of the target gene and an antisense strand which includes a complementary sequence to the sense strand, is introduced into a cell or the like to cause the degradation or cleavage of the mRNA of the target gene. The RNAi phenomenon has been observed in various species, such as, but not limited to, flies, insects, protozoa, vertebrates, and higher plants. There are reports on a mechanism of action of the RNAi phenomenon at a molecular level through many studies on Drosophila and Caenorhabditis elegans. For example, a small RNA fragment called small interfering RNA (siRNA) is an essential, sequence-specific mediator of the RNAi phenomenon (Hammond, S. M. et al., Nature, Vol. 404, pp. 293-296, 2000; Parrish, S. et al., Mol. Cell, Vol. 6, pp. 1077-1087, 2000; Zamore, P. D. et al., Cell, Vol. 101, pp. 25-33, 2000); and siRNA is produced from a long double-stranded RNA with a RNase III-like nuclease called Dicer (Brenstein, E. et al., Nature, Vol. 409, pp. 363-366,2001; Elbashir, S. M. et al., Genes Dev, Vol. 15, pp. 188-200, 2001). siRNA is expected to be applied to a therapy for a dermatosis, or the like, as a functional nucleic acid capable of suppressing the expression of a target gene with ease.

Methods of introducing a functional nucleic acid intradermally, by applying the functional nucleic acid on the skin of an organism or by an intradermal injection is generally known. However, intradermal permeability of a functional nucleic acid by application to the skin is extremely low.

Methods such as electroporation have been developed as means for promoting intake of a nucleic acid into a cell. However, in the electroporation, though momentary application, it is necessary to apply a current at high voltage to the skin of a patient. Accordingly, there has been a concern for physical injury to the cells which form the skin, so electroporation is problematic from the viewpoint of safety and quality of life (QOL) of patients.

On the other hand, a method of introducing, i.e. permeating, an ionic drug, such as a functional nucleic acid having a net charge, through the skin or the mucosa (herein collectively referred to as “skin”) of an organism by applying an electromotive force to the ionic drug, which has been applied in a predetermined portion to the skin of an organism, is called iontophoresis (see, for example, JP 63-35266 A). In recent years, iontophoresis has been disclosed as a noninvasive and safe method for administering a drug to an organism.

In iontophoresis, an ionic compound having a positive charge is generally driven, i.e., transported through and/or into the skin of an organism by repulsion by the anode side of the electromotive force. On the other hand, an ionic compound having a negative charge is generally driven, i.e. transported through and/or into the skin of an organism by repulsion by the cathode side of the electromotive force.

However, it is known that functional (e.g., biologically active) nucleic acids have a relatively high molecular weight and are generally not suitable for transdermal or intradermal administration (see, for example, Kikuchi, Y. et al., Journal of Investigative Dermatology, (2007), Vol. 127, No. 11, pp. 2577-2584). It has been clarified in the experiments by the inventors named in the present application that, due to inhibition by the barrier function of the horny layer of skin, it is difficult to deliver an effective dose of an siRNA intradermally (an “effective dose” is one that is capable of suppressing the expression of a target gene) even if the siRNA is administered alone by iontophoresis.

### BRIEF SUMMARY

Disclosed herein are methods of intradermally administering to an organism an siRNA efficiently by iontophoresis, wherein the siRNA effectively suppresses the expression of a target gene.

The inventors named in the present application have found that, by administering a negatively-charged complex formed from an siRNA and a polycation to an organism by iontophoresis, it is possible to efficiently deliver the siRNA intradermally into the skin and to suppress the expression of a target gene effectively. The embodiments of the present application are based on these findings.

Accordingly, one embodiment is to provide a composition for iontophoresis that is capable of efficiently delivering an siRNA into the skin of an organism wherein the siRNA delivered effectively suppresses the expression of a target gene.

Such a composition for iontophoresis comprises an siRNA-polycation complex which has a negative charge.

Another embodiment is to provide an siRNA-polycation complex which is administered to an organism by iontophoresis, wherein the administration of the siRNA-polycation complex efficiently delivers the siRNA into the skin of the organism (i.e., intradermally) by passing through the horny barrier of the skin and by effectively suppressing the expression of a target gene.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic drawing illustrating an iontophoresis device used for administering an siRNA-protamine complex or an siRNA-liposome complex in an in vivo test according to one illustrated embodiment.

FIG. 2A is a photograph with an inverted confocal laser scanning microscope of a frozen skin sample of a rat after administration of only siRNA by iontophoresis.

FIG. 2B is a photograph with an inverted confocal laser scanning microscope of a frozen skin sample of a rat after administration of a Cy3-labeled siRNA-protamine complex by iontophoresis.

FIG. 3 shows results of PCR quantitative determination of an MAPK-1 mRNA in the case where a MAPK-1 siRNA-protamine complex was administered to a rat by iontophoresis according to one illustrated embodiment.

FIG. 4 is a photograph with a inverted confocal laser scanning microscope of a frozen skin sample of a rat after administration of a Cy3-labeled siRNA-liposome complex by iontophoresis according to one illustrated embodiment.

FIG. 5 shows a PCR quantitative determination of an MAPK-1 mRNA in the case where an MAPK-1 siRNA-liposome complex was administered to a rat by iontophoresis according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with intradermal delivery devices have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

As used herein and in the claims, the term “siRNA” refers to small interfering RNA. An siRNA is a double-stranded RNA which is capable of inducing suppression of the expression of a target gene by the RNAi phenomenon and which comprises 15 to 30 base pairs (bp). The number of nucleotides of in each of the single-stranded RNA's forming the double-stranded RNA may be the same or different from each other. The siRNA may comprise a part formed of a single strand part (referred to herein in the singular as an “overhang”).

When the siRNA comprises an overhang, the full length of the siRNA is represented by the sum of the length of the paired central double-stranded part and the length of the overhangs protruding at both ends of the central paired double-stranded portion (the overhangs are assumed to form a pair). For example, when there is a central double-stranded part of 19 base pairs (bp) and overhangs of 4 bp at both ends, the full length of the siRNA is represented as 23 bp.

The term “double-stranded part” means a part at which nucleotides of both strands pair with each other in siRNA, that is, a part in the siRNA excluding the single-stranded part.

The term “sense strand” means a nucleotide strand having a sequence homologous to a coding strand of the target gene.

The term “antisense strand” means a nucleotide strand having a sequence complementary to a coding strand of a gene. That is, the “antisense strand” is a strand having a complementary sequence to an mRNA of the target gene, and the “antisense strand” may bind to the mRNA of the target gene to induce the RNAi phenomenon. The “antisense strand” and “sense strand” anneal to produce an siRNA.

The term “complementary” means that two nucleotides (bases) are capable of pairing with each other under a hybridization condition, and refers to a relationship between adenine (A) and thymine (T), or between adenine (A) and uracil (U), and between cytosine (C) and guanine (G), for example.

The term “polycation” means a substance which is formed of two or more constitutional units having positive charges and has a net positive charge at a selected pH, such as a physiological pH. The “polycation” may be formed of a uniform constitutional unit or two or more different constitutional units.

The term “cationic protein” means a protein formed of two or more amino acids having positive charges and which has a net positive charge at a selected pH, such as a physiological pH. The term “protein” includes peptides (oligomers) and polypeptides.

The term “cationic liposome” means a liposome having a net positive charge in a selected pH, such as a physiological pH.

The term “cationic lipid” means a lipid having a net positive charge in a selected pH, such as a physiological pH. The term “neutral lipid” means a lipid means a lipid having a no net positive or negative charge and can be being present in the form of a non-charge or a neutral amphoteric ion at a selected pH, such as a physiological pH.

### Composition for Iontophoresis

A composition useful in intradermally delivering an siRNA to an organism by iontophoresis comprises a negatively-charged siRNA-polycation complex. When the complex formed of an siRNA and a polycation is administered to an organism by iontophoresis, the siRNA is efficiently delivered to (i.e., administered to) the corium layer of the skin of the organism by passing through the horny layer of the skin, the epidermal layer of the skin, and the basal membrane layer of the skin, and hence the expression of a target gene within the corium layer can be effectively suppressed by the siRNA. A composition useful in intradermally delivering an siRNA to an organism by iontophoresis may comprise components in addition to the negatively-charged siRNA-polycation complex.

siRNA-Polycation Complex

An siRNA-polycation complex is characterized in that the complex is formed of an siRNA and a polycation, and the net charge of the whole complex is negative. The siRNA-polycation complex may be formed by placing the siRNA and the polycation in a system capable of generating a charge interaction and aggregating the siRNA and the polycation to form the siRNA-polycation complex. In general, the siRNA-polycation complex is formed by binding of the siRNA and the polycation using an electrostatic interaction as a main driving force.

A negatively-charged siRNA-polycation complex has a zeta potential of preferably −50 to −5 mV and more preferably −30 to −10 mV.

An siRNA-polycation complex can be formed efficiently by adjusting a negative to positive (−/+) charge ratio of the siRNA to the polycation and mixing the siRNA and the polycation together. In the siRNA-polycation complex, the −/+charge ratio of the siRNA to the polycation is preferably 2:1 to 7:3 and more preferably 2:1 to 6:4. In the case of formation of the siRNA-polycation complex, the mixing ratio of the siRNA and the polycation can be appropriately adjusted based on the charge ratio.

An siRNA-polycation complex has preferably a particle-like form. The average particle diameter of an siRNA-polycation complex is not particularly limited as long as the siRNA can be delivered intradermally by iontophoresis, and is preferably from 50 nm to 3,000 nm and more preferably from 50 nm to 1,000 nm. As a method of determining the average particle diameter, there are given dynamic light scattering, static light scattering, observation with an electromicroscope, and observation with an atomic force microscope, for example.

siRNA

An siRNA of the siRNA-polycation complex is easily negatively charged because the nucleotides of the constitutional unit of an siRNA have negative charges, thereby allowing the formation of a complex with a polycation by electrostatic interaction.

The total number of negative charges in an siRNA of the invention is preferably from 30 to 60 and more preferably from 36 to 54. The total number of the negative charges can be calculated, for example, from the total number of nucleotides forming both strands of the siRNA.

An siRNA may have a double-stranded part, and overhangs and/or an overhang at the 5′ or 3′ terminal of the sense strand and/or the antisense strand as long as the siRNA can induce the suppression of the expression of a target gene by RNAi. In one embodiment, the siRNA has a double-stranded part, and overhangs and/or an overhang at the 3′ terminal of the sense strand and/or the antisense strand.

The number of the nucleotides of each sense strand and antisense strand which form an siRNA is preferably from 15 to 30 and more preferably from 18 to 27. In addition, the number of the nucleotides of the sense strand and the antisense strand may be the same as each other or different from each other, and preferably the same as each other.

The length of the double-stranded part of an siRNA may be, for example, from 15 to 30 bp, preferably from 18 to 27 bp, and more preferably from 19 to 27 bp.

Each overhang in an siRNA is arbitrarily formed of one or two nucleotides (ribonucleic acid or deoxyribonucleic acid) and more preferably of two nucleotides. In addition, the nucleotide in an overhang is preferably complementary to the nucleotide sequence of the target gene and may be not complementary to the nucleotide sequence of the target gene.

As the ribonucleic acid forming an overhang of the siRNA, U (uridine), A (adenine), G (guanosine), or C (cytidine), for example, may be used. As the deoxyribonucleic acid forming the overhang, dT (deoxythymidine), dA (deoxyadenosine), dG (deoxyguanosine), or dC (deoxycytidine), for example, may be used.

According to one embodiment, the overhang is one in which one or two bases of U or dT are added at the 3′ terminals of the sense strand and/or the antisense strand independently. According to one embodiment, the overhang is one in which one or two bases of U or dT are added at the 3′ terminals of the sense strand and the antisense strand independently. According to one embodiment, the overhang is one in which two bases of dT are added at the 3′ terminals of the sense strand and the antisense strand.

An siRNA is not particularly limited as long as the siRNA is designed based on a DNA base sequence specific to the target gene and can suppress the expression of the target gene. For example, the siRNA can be designed and produced by a conventional method (see, for example, Caplen, N. J. et al., “Specific inhibition of gene expression by small double-stranded RNAs in invertebrate and vertebrate systems”, PNAS (Proceedings of The National Academy of Sciences USA), Vol. 98, No. 17, pp. 9742-9747, 2001 (August 14); Elbashir, S. M., et al., “Duplexes of 21±nucleotide RNAs mediate RNA interference in cultured mammalian cells”,Nature, Vol. 411, pp. 494-498, 2001. (May 24); Harborth, J. et al., “Identification of essential genes in cultured mammalian cells using small interfering RNAs”, Journal of Cell Science, Vol. 114, No. 24, pp. 4557-4565, 2001.)

An siRNA may have a stem-loop structure in which the terminal parts of two RNA strands are connected to each other with a linker sequence. An siRNA having a stem-loop structure can be easily produced, for example, by constructing and expressing a vector capable of expressing a stem-loop siRNA. The vector capable of expressing an siRNA having a stem-loop structure can be easily constructed appropriately by a person skilled in the art (see, for example, Bass, B. L., Cell, Vol. 101, pp. 235-238, 2000; Tavernarakis, N. et al., Nat. Genet., Vol. 24, pp. 180-183, 2000; Malagon, F. et al., Mol. Gen. Genet, Vol. 259, pp. 639-644, 1998; and Parrish, S. et al., Mol. Cell, Vol. 6, pp. 1077-1087, 2000).

### Target Gene

A target gene is a gene whose expression can be suppressed by the RNAi phenomenon generated by an siRNA of the invention. A target gene may be arbitrary selected. The target gene can be a gene such that the genome sequence of the gene has been determined or the functions of the gene may be desired to be clarified. The target gene can be a gene whose expression may cause a disease, or the like. A gene whose genome sequence has not yet been determined may be selected as the target gene as long as at least a sequence of 15 or more nucleotides, which is the length capable of binding to a part of the mRNA sequence of the gene, that is, the other strand (antisense strand) of the siRNA, has been determined. Therefore, a gene where part of its mRNA sequence has been determined, but not where the whole sequence of the gene has been determined, may also be selected as a “target gene”.

### Polycation

The polycation forms, with the siRNA, a negatively-charged siRNA-polycation complex in a system capable of generating electrostatic charge interaction.

The structure of the polycation is not particularly limited as long as the polycation can form the siRNA-polycation complex which can be delivered intradermally to an organism by iontophoresis, and may be a chain-like, branched-like, cyclic, or spherical shape, or an acinous shape having a void inside.

In addition, the polycation may be one of a natural product or a synthetic product as long as the polycation has a net positive charge, and may have a form of a polymer, liposome, or fatty acid-binding peptide, and is preferably a cationic protein or a cationic liposome.

### Cationic Protein

A cationic protein useful as a polycation in the siRNA-polycation complex may be a protein formed of the same kinds of amino acids having positive charges or a protein formed of two or more different kinds of amino acids having positive charges. In addition, the cationic protein may include both amino acids having positive charges and amino acids having negative charges, as long as the cationic protein has a net positive charge.

The total number of positive charges in the cationic protein is preferably from 1 to 100 and more preferably from 5 to 30. The total number of positive charges can be determined from a difference between the number of amino acids having positive charges and the number of amino acids having negative charges in the cationic protein.

In the case of using a cationic protein as the polycation in the negatively-charged siRNA-polycation complex, the −/+charge ratio of the siRNA to the cationic protein in the negatively-charged siRNA-polycation complex can be calculated by the following equation.

(−/+charge ratio)=[(siRNA amount (mol))×(the total number of negative charges in siRNA)]:[(cationic protein amount (mol))×(the total number of positive charges in the cationic protein)] Equation 1

The siRNA amount and the cationic protein amount can be easily determined by one skilled in the art in view of a loading amount upon preparation of the complex.

The molecular weight of the cationic protein is not particularly limited as long as the cationic protein can form the negatively-charged siRNA-polycation complex, and is preferably from 500 to 50,000 kDa and more preferably from 1,000 to 10,000 kDa.

Examples of cationic proteins include, but are not particularly limited as long as the cationic protein has a net positive charge, uniform polycations such as polylysine, polyarginine, and polyornithine, natural polycationic DNA-binding proteins such as histone and protamine, analogues or fragments thereof. Preferred cationic proteins are protamine, poly-L-lysine, an arginine oligomer, or a lysine oligomer.

### Cationic Liposome

A cationic liposome useful as a polycation in the siRNA-polycation complex also has a net positive charge and can form a negatively-charged siRNA-polycation complex with an siRNA.

In the case of using a cationic liposome as the polycation in the negatively-charged siRNA-polycation complex, the −/+charge ratio of negative charges in the siRNA to positive charges in the cationic liposome is calculated based on the following principal: half of the total charge of the cationic liposome is used as the charge of the cationic liposome because the liposome is formed of a lipid bilayer and the positive charge inside the bilayer is not involved in electrostatic interaction with the siRNA. For example, the liposome is formed of a cationic lipid having a monovalent positive charge, the −/+charge ratio can be calculated by the following equation.

(−/+charge ratio)=[(siRNA amount (mol))×(the total number of negative charges in siRNA)]:[(cationic lipid amount (mol))/2] Equation 2

The siRNA amount and the cationic liposome amount can be easily determined by one skilled in the art in view of a loading amount upon preparation of the complex.

The average particle diameter of a cationic liposome is not particularly limited as long as the cationic liposome can form a negatively-charged siRNA-polycation complex and can be delivered intradermally, and is preferably from 50 nm to 1,000 nm and more preferably from 100 nm to 500 nm. A method of determining the average particle diameter is the same as the method of determining the particle diameter of the complex.

A cationic liposome includes at least a cationic lipid as a constitutional component. The cationic lipid is preferably a C12-C20 lipid having a positive charge of 1 to 10 valences, more preferably a C14-C20 lipid having a positive charge of 1 to 3 valences, and more preferably a C14-C18 lipid having a positive charge of 1 valence. Examples of cationic lipids include, but are not limited to, 1,2-dioleoyloxy-3-(trimethylammonium)propane (DOTAP), dioctadecyldimethyl ammonium chloride (DODAC), N-(2,3-dioleyloxy)propyl-N,N,N-trimethyl ammonium (DOTMA), didodecylammonium bromide (DDAB), 1,2-dimyristoyloxypropyl 1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are 1,2-dioleoyloxy-3-(trimethylammonium)propane(DOTAP), dioctadecyldimethyl ammonium chloride (DODAC), N-(2,3-dioleyloxy)propyl-N,N,N-trimethyl ammonium (DOTMA), and 2,3-dioleoyloxy-N-[2(spermine carboxamide)ethyl]-N,N-dimethyl-1-propanamium trifluoroacetate (DOSPA). More preferred is 1,2-dioleoyloxy-3-(trimethylammonium)propane(DOTAP).

In addition, the liposome preferably further includes a neutral lipid as another component of the cationic liposome, in view of structural stability and the like.

The neutral lipid can be appropriately selected in view of the delivery efficiency of the siRNA-polycation complex, and is preferably a C12-C20 neutral lipid and more preferably a C14-C18 neutral lipid. Examples of neutral lipids include, but are not limited to, diacylphosphatidyl choline, diacylphosphatidyl ethanol amine, ceramide, sphingoemyelin, cephalin, sterol, and cerebroside. Preferred is diacylphosphatidyl ethanol amine or diacylphosphatidyl choline. More preferred is dioleylphosphatidyl ethanol amine.

In the case where a cationic liposome includes both a cationic lipid and a neutral lipid, the molar ratio of the cationic lipid to the neutral lipid can be appropriately determined in view of stability of the liposome and the like, and is preferably from 2:8 to 8:2, and more preferably from 3:7 to 7:3.

A cationic liposome as described above can be prepared by known techniques using a cationic lipid and a neutral lipid. For example, a cationic lipid, a neutral lipid, and the like, are mixed at a desired ratio in a liquid medium such as water, whereby a mixed solution is obtained. Next, the medium is removed under reduced pressure, and thus a lipid membrane is obtained. Next, a buffer is added to the lipid membrane, and the obtained membrane is hydrated in the obtained mixed liquid. Further, the mixed liquid is subjected to sonication, and treated with a membrane filter as required to adjust particle diameters, whereby a cationic liposome is obtained.

An siRNA-polycation complex can be used as a composition for iontophoresis in the methods disclosed herein. The composition may comprise one or more components in addition to the siRNA-polycation complex as long as the additional components do not prevent the administration of the complex by iontophoresis.

The additional components are not particularly limited as long as the additional components do not prevent administration of the complex by iontophoresis. Examples of additional components include, but are not limited to, water and pharmaceutically acceptable carriers, such as buffering agents such as HEPES, preservatives, solubilizing agents, antiseptic agents, stabilizers, antioxidants, and colorants and the like. Further, the composition comprising the siRNA-polycation complex may be formed into an appropriate formulation as desired as long as administration of the complex by iontophoresis is not prevented. For example, the composition can be formed into a dried form. However, in view of efficient administration of the complex by iontophoresis, the composition is preferably formed into a solution or a suspension in combination with water or a HEPES buffer. In this case, the pH of the composition for iontophoresis may be, for example, a physiological pH or the like, and, more specifically, a pH of from 7 to 8. In addition, the ionic strength of the composition is from 5 to 20 mM, for example.

The content of the siRNA-polycation complex in the composition may be appropriately determined by one skilled in the art as required.

### Production Method

An siRNA-polycation complex can be easily formed by mixing an siRNA and a polycation in a system in which charge interaction may be generated. The details of the production method of the siRNA-polycation complex are as follows.

In the production method of the siRNA-polycation complex, a first aqueous solution containing an siRNA and a second aqueous solution containing a polycation are prepared.

The concentration of the siRNA in the first aqueous solution and the concentration of the polycation in the second aqueous solution are appropriately determined by one skilled in the art in view of solubility of each siRNA and polycation, formation efficiency of the siRNA-polycation complex, and the like.

Furthermore, the pH, ion strength, and temperature of the first and second aqueous solutions may be appropriately adjusted by one skilled in the art in view of the charging states of the siRNA and the polycation, and formation efficiency of the final complex.

Furthermore, the first and second aqueous solutions may preferably include water or buffer, more preferably, water or HEPES buffer, and the like. Next, the first aqueous solution and the second aqueous solution are mixed together to obtain an siRNA-polycation complex.

The mixing method is not particularly limited. The second aqueous solution may be added to the first aqueous solution, and alternatively, the first aqueous solution may be added to the second aqueous solution. In addition, the first aqueous solution and the second aqueous solution may be added to a container simultaneously to be mixed together. The thus obtained mixed liquid of the first aqueous solution and the second aqueous solution may be stirred appropriately.

The mixing ratio of the first and the second aqueous solutions may be appropriately determined by one skilled in the art so that an siRNA-polycation complex having excess negative charge is formed in view of the −/+charge ratio of the siRNA and the polycation in the mixed liquid. For example, the mixing ratio may be set so that the total number of the negative charges of the siRNA in the first aqueous solution is in excess of the total number of positive charges of the polycation in the second aqueous solution.

In addition, the pH, ion strength, and temperature of the mixed liquid containing the siRNA and the polycation may be appropriately determined by one skilled in the art in view of the formation efficiency of the complex. The pH and ion strength of the mixed liquid can be adjusted by one skilled in the art by changing the compositions (concentration amount, pH, and ionic strength) and the mixing ratios of the first aqueous solution and the second aqueous solution beforehand. The pH of the mixed liquid is 3 to 10, for example. In addition, the ionic strength of the mixed liquid is 5 nM to 20 mM, for example.

The temperature of the mixed liquid is from 16° C. to 40° C., for example.

### Application

The siRNA-polycation complex, and compositions thereof, is applied to an organism by iontophoresis. Therefore, one embodiment is a use of a negatively-charged siRNA-polycation complex in a production of the composition for iontophoresis. A composition for iontophoresis of the invention can efficiently deliver an siRNA intradermally and effectively suppress the expression of a target gene by the RNAi phenomenon. Accordingly, a composition of the invention can be used as a drug such as a gene expression suppressor. Further, in the case where a target gene is known as or is determined to be a disease-related gene, a novel composition described in this application can be favorably used as an agent for a gene therapy of the disease.

### Electrode Assembly and Iontophoresis Device

In addition, administration of the siRNA-polycation complex to an organism can be favorably performed by using an electrode assembly which holds the composition for iontophoresis and an iontophoresis device equipped with the electrode assembly.

According to one embodiment, provided is an electrode assembly for iontophoresis including an electrode, an siRNA-polycation complex holding portion for holding the composition, the holding portion being placed in a skin side of the electrode, in which the siRNA-polycation complex can be released to an organism skin by iontophoresis. In addition, for example, an electrolyte solution holding portion for holding the electrolyte may be further provided between the electrode and the siRNA-polycation complex holding portion as long as administration of the siRNA-polycation complex by iontophoresis is not prevented.

In addition, the siRNA-polycation complex is charged negatively, so a negative current is preferably applied to a cathode side of an electric system. Therefore, in the electrode assembly, the electrode refers to a cathode electrode. In addition, an electrode formed of a conductive material such as carbon or platinum is preferably used. Those materials can be also used in the counter electrode as described below.

The siRNA-polycation complex holding portion may be formed of a cell (electrode chamber) made of acryl or the like, which is filled with the composition. Alternatively, the siRNA-polycation complex holding portion may be formed of a non-woven fabric, an absorbent cotton, or a thin membrane body, which characteristics of impregnating and holding the composition. As a constitutional member of the thin membrane body, a material having both a favorable impregnation-holding characteristic and a favorable ion delivery property is preferred. Examples of the material include a hydrogel body of an acrylic resin (acrylic hydrogel membrane) and a segmented polyurethane-based gel membrane. The above cell and thin membrane body can be also used in the constitution of the electrolyte solution holding portion.

In addition, the constitution of the iontophoresis device can be appropriately changed as long as the iontophoresis device includes the above electrode assembly and is capable of intradermally administering the siRNA-polycation complex. The iontophoresis device preferably includes at least an electric source unit, the electrode assembly connected to a cathode of the electric source unit, and an electrode assembly connected to an anode of the electric source unit.

The structure of the electrode assembly connected to the anode can be appropriately changed as long as the electrode assembly can function as a counter electrode of the electrode assembly which holds the siRNA-polycation complex. For example, the electrode assembly as a counter electrode may have the same constitution as that of the electrode assembly connected to an anode except that the siRNA-polycation complex holding portion is changed to an electrolyte solution holding portion. According to one embodiment, the electrode assembly as a counter electrode includes at least an anode electrode. In addition, according to a preferred embodiment, the electrode assembly as a counter electrode further includes the electrolyte solution holding portion which is provided to a skin side of the anode electrode and holds an electrolyte solution.

Method of Intradermally Administering an siRNA/Method of Suppressing Expression of Target Gene

An siRNA can be efficiently administered intradermally to an organism as described above via the siRNA-polycation complex. Therefore, according to one embodiment, provided is an intradermal administration method for an siRNA, including intradermally administering a negatively-charged siRNA-polycation complex to the skin of an organism by iontophoresis.

In the case where the siRNA-polycation complex is administered to the skin of an organism by iontophoresis, the expression of a target gene can be suppressed effectively by the RNAi phenomenon as mediated by the siRNA in the complex. Therefore, according to one embodiment, provided is a method of suppressing the expression of a target gene in an organism by the RNAi phenomenon which includes administering an effective dose of an siRNA-polycation complex to an organism by iontophoresis.

When the expression of the target gene is involved in the pathogenesis or progress of a disease, the above methods can be favorably used in therapy for the disease. Examples of the disease are not particularly limited, and include atopic dermatitis (AD), skin cancer, and skin infections such as psoriasis, candida, and dermatomycosis.

In the iontophoresis, energization conditions may be appropriately determined in view of administration efficiency of the siRNA-polycation complex. The current value is preferably from 0.1 to 0.45 mA/cm² and more preferably from 0.1 to 0.2 mA/cm².

The effective dose of an siRNA-polycation complex is appropriately determined by a person skilled in the art in view of the kind of disease, species, gender, age, body weight, and state of the organism, administration plan, and the like.

An “organism” is preferably a mammalian animal, preferably a human, cow, pig, horse, sheep, dog or cat, and still more preferably a human.

Hereinafter, embodiments of the application are described in detail by way of several examples. It is understood that the invention is not limited by these examples.

### TEST EXAMPLE 1

### Studies on Zeta Potential and Particle Diameter of siRNA-Protamine Complex

Preparation of siRNA-Protamine Complex

A Cy3-labeled siRNA (negative control siRNA purchased from Ambion, Inc.) and protamine were mixed using a vortex mixer in distilled water at room temperature at a mixing ratio by weight shown in Table 1 below. The obtained mixed liquid was left at rest at room temperature for 10 minutes, whereby a suspension containing a Cy3-labeled siRNA-protamine complex was obtained.

Next, the suspension containing the siRNA-protamine complex was filled in a dedicated cuvette, and the particle diameter and zeta potential were measured by a Zetasizer®.

The results are as shown in Table 1. In the case where the siRNA and the protamine were mixed at a mixing ratio by weight of 5:1, the zeta potential of the siRNA-protamine complex represented a negative charge. In this case, the −/+charge ratio of the siRNA to the protamine was calculated to be 3:1. In addition, the siRNA-protamine complex had an average particle diameter of 93.3 nm and the complex having a particle diameter of around 200 nm accounted for about 60%.

TABLE 1
Mixing ratio by weight of siRNA-protamine
and physical properties of complex

| Mass ratio | Average particle | Zeta potential |
|---|---|---|
| (siRNA:protamine) | diameter (nm) | (mV) |
| 2:3 | 262 | 25.1 |
| 1:1 | 254 | 23.5 |
| 2:1 | 94.8 | −3.3 |
| 3:1 | 1,650 | −4.2 |
| 4:1 | 1,030 | −3.98 |
| 5:1 | 93.3 | −11.9 |

* Values in Table 1 represent average values.

### TEST EXAMPLE 2

### Iontophoresis

Preparation of Fluorescence-Labeled siRNA-Protamine Complex

About 10 μg (100 μl) of a Cy3-labeled siRNA (negative control siRNA purchased from Ambion Inc.) and 100 μl of a 0.02 mg/ml protamine solution (containing about 2 μg of protamine, solvent: distilled water) were mixed at room temperature using a vortex mixer. The obtained mixed liquid was left at rest at room temperature for 10 minutes, whereby a suspension containing the Cy3-labeled siRNA-protamine complex was obtained.

### Iontophoresis Device

The Cy3-labeled siRNA-protamine complex was formed of the same mixing ratio by weight as that in Test Example 1 and was charged negatively, thereby being administered from a cathode side. The iontophoresis device used in the administration of the Cy3-labeled siRNA-protamine complex is as shown in FIG. 1.

In FIG. 1, an iontophoresis device 1 is placed on a skin 5 and is configured of an electric source unit 2, an electrode assembly 3 for holding the complex, and an electrode assembly 4 as a counter electrode thereof. Those electrode assemblies are connected with electrically conductive paths 6 and 7. The electrode assembly 3 was formed of a cathode electrode 31, an siRNA-polycation complex holding portion 32 provided at a skin side of the cathode electrode 31. On the other hand, the electrode assembly 4 was formed of an anode electrode 41, an electrolyte holding portion 42 which was provided at a skin side of the anode electrode 41 and held 200 μl of an electrolyte solution. The siRNA-polycation complex holding portion 32 and electrolyte solution holding portions 42 each were formed of a cell (electrode chamber) that can contain an electrolyte solution or the like. In addition, the siRNA-polycation complex holding portion 32 and the electrolyte solution holding portion 42 each were formed of a non-woven fabric or absorbent cotton impregnated with an siRNA-polycation complex solution or an electrolyte solution.

Iontophoresis of siRNA-Protamine Complex

An siRNA holding portion of an iontophoresis device was filled with 200 μl of the Cy3-labeled siRNA-protamine suspension and the device was mounted on a back skin of an SD rat (n=1) shaved with clippers under Nembutal® anesthesia. Then, the suspension (Cy3-labeled siRNA-protamine complex) was administered at a constant current of 0.45 mA (0.15 mA/cm²) for 60 minutes by iontophoresis.

In addition, 200 μl of Cy3-labeled siRNA solution (siRNA: about 10 μg, solvent: distilled water) were used instead of the siRNA-protamine suspension as a control. According to the same procedure as described above, the Cy3-labeled siRNA was administered by iontophoresis.

### Preparation of Skin Slice and CLSM Observation

The skin was cut out immediately after the completion of iontophoresis, and the resultant was frozen and embedded in an Optimal Cutting Temperature (OCT) compound. A slice measuring 15 μm was prepared from the frozen skin sample with a cryostat and observed with an inverted confocal laser scanning microscope (LSM510 Carl Zeiss).

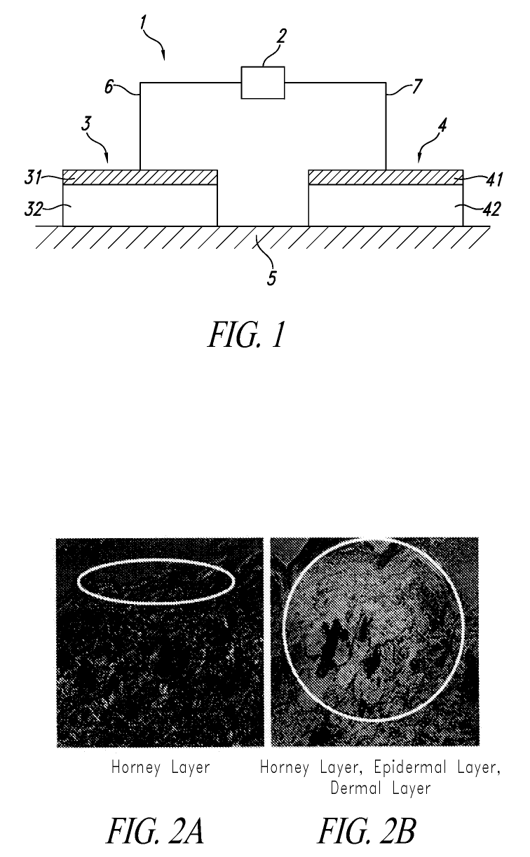

A result was as shown in FIG. 2A. In the case where only Cy3-labeled siRNA was administered, no fluorescence was observed in an epidermal layer or a dermal layer except for a horny layer (FIG. 2A). On the other hand, in the case where the Cy3-labeled siRNA-protamine complex was administered, fluorescence was observed in both an epidermal layer and dermal layer, and hence the Cy3-labeled siRNA-protamine was confirmed to be delivered to the epidermal layer and dermal layer by passing through the horny layer.

### TEST EXAMPLE 3

### Evaluation for Effect of Suppressing Gene Expression by siRNA-Protamine Complex

In order to evaluate an effect of suppressing a gene expression by an intradermal delivery of an siRNA-protamine complex, Mitogen-activated protein kinase 1 (MAPK-1) was selected as an objective gene, which has been known to express in almost all cells of an organism, and experiments were then performed according to the following procedure.

Preparation of MAPK-1 siRNA-Protamine Complex

10 μg (100 μl) equivalent of a MAPK-1 siRNA (purchased from QIAGEN) and 100 μl of a 0.01 mg/ml protamine solution (equivalent to 1 μg of protamine, solvent: distilled water) were mixed at room temperature using a vortex mixer. The obtained mixed liquid was left at rest at room temperature for 10 minutes, whereby a suspension containing the siRNA-protamine complex was obtained.

The base sequences of the sense strand and antisense strand of the MAPK-1 siRNA, and the amino acid sequence of the protamine were as follows.

Base Sequence of MAPK-1 siRNA-Protamine Complex

| | |
|---|---|
| Sense strand: | |
| CCCUCACAAGAGGAUUGAATT | (SEQ ID NO: 1) |
| Antisense strand: | |
| UUCAAUCCUCUUGUGAGGGTT | (SEQ ID NO: 2) |
| Protamine: | |
| PRRRRSSSRPVRRRRRPRVSRRRRRRGGRRRR | (SEQ ID NO: 3) |

Iontophoresis of MAPK-1 siRNA-Protamine Complex

According to the same method as in Test Example 2, the MAPK-1 siRNA-protamine complex was administered to a back skin of an SD rat by iontophoresis.

Quantitative Determination of MAPK-1 mRNA

Forty-eight hours after the iontophoresis, the rat was euthanized under anesthesia, and the part of the skin at which iontophoresis was conducted was extirpated. The adipose tissue adhered to the extirpated skin was excised, and an RNA was extracted and purified. As a control, an RNA was extracted and purified from a skin of an untreated rat in the same manner. After the extracted RNA sample was reverse-transcribed by using a random primer (9 mer), PCR was conducted using a primer for detecting MAPK-1 having the following base sequence specific to MAPK-1.

### PCR Primers for Detecting MAPK-1

| | | |
|---|---|---|
| Forward: | AGTTGAACAGGCTCTGGCCCA | (SEQ ID NO: 4) |
| Reverse: | TGAACGCGTCCAGTCCTCTGA | (SEQ ID NO: 5) |

The PCR product was separated by a 2% agarose gel electrophoresis, stained with SYBR Green, detected a UV transilluminator and photographed. The photographed electrophoresis image was analyzed with an ATTO CS Analyzer 2.0 software and the band intensities were measured. 18S rRNA (a primer for 18S rRNA was purchased from Ambion, Inc.) was used as an internal control for quantification. Accordingly, the band intensity of MAPK-1 was evaluated as a relative expression level by dividing the band intensity of MAPK-1 by the band intensity of the 18S rRNA.

With respect to the untreated group to which no siRNA-protamine complex was administered, PCR was conducted and the band intensity of MAPK-1 was measured as a control in the same manner as described above.

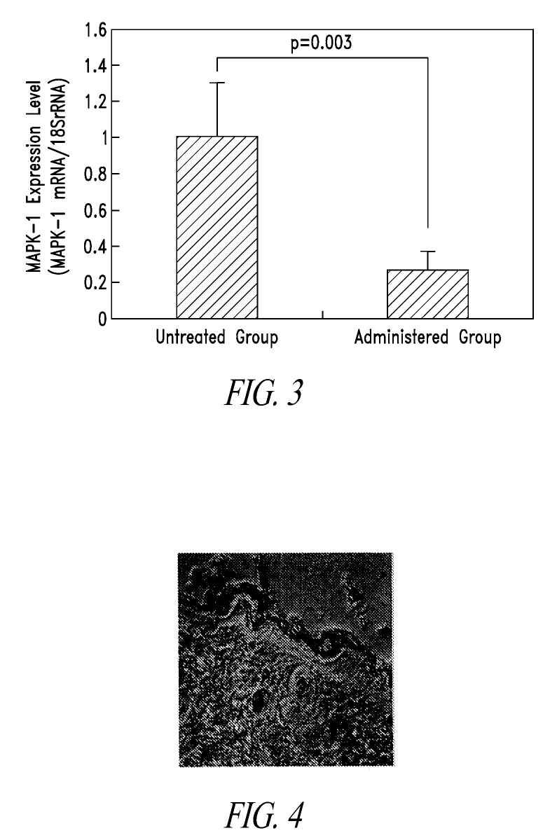

The results were as shown in FIG. 3. The MAPK-1 mRNA amount in the skin of the siRNA-protamine complex administration group decreased to about 28% of the untreated group. Further, the MAPK-1 mRNA amount of the siRNA-protamine complex administration group was significantly low compared to that of the untreated group (name of statistical test, Student's t-test).

### TEST EXAMPLE 4

### Study on Zeta Potential and Particle Diameter of siRNA-Liposome Complex

### Preparation of Cationic Liposome:

152 μg of a cationic lipid (DOTAP) and 30.4 μg of a neutral lipid (DOPE) (mixing ratio by weight of DOTAP:DOPE was 5:1, molar ratio of DOTAP:DOPE was 11:2) were mixed in an organic solvent such as CHCl₃, whereby a solution was obtained. After the solvent was distilled off under reduced pressure, addition of the organic solvent and distillation under reduced pressure were repeated, and thus a lipid membrane was prepared. Next, the lipid membrane was hydrated with 100 μl of sterile distilled water (DDW), and sonicated for 3 minutes, whereby a liposome suspension containing a cationic liposome was obtained.

Preparation of siRNA-Liposome Complex

To a solution (100 μl, solvent: distilled water) containing 10 μg of MAPK-1 siRNA, the liposome suspension was added. The resultant solution was mixed with a vortex, and a suspension containing an siRNA-liposome complex was obtained. In this time, the mixing ratio by weight of the MAPK-1 siRNA to the liposome was adjusted according to description in Table 2 below.

Measurement of Particle Diameter and Zeta Potential of siRNA-Liposome Complex

The suspension containing the siRNA-liposome complex was filled in a dedicated cuvette, and the particle diameter and zeta potential were measured with a Zetasizer®.

The results were as shown in Table 2.

TABLE 2
Mixing ratio and particle diameter of siRNA-liposome complex

| Mass ratio | | | |
|---|---|---|---|
| (siRNA:total | Mass ratio | Average particle | Zeta potential |
| lipid) | (siRNA:DOTAP) | diameter (nm) | (mV) |
| 10:91.2 | 10:76 | 148 | 49.3 |
| 10:36.48 | 10:30.4 | 167 | 45.9 |
| 10:18.24 | 10:15.2 | 84.2 | −30.2 |
| 10:9.12 | 10:7.6 | 68.2 | −28.7 |

In the case where the mixing ratios by weight of siRNA:DOTAP were 10:15.2 and 10:7.6, the zeta potential (surface potential) of the siRNA-liposome complex was negative charge. In this case, the −/+ratios of the negative charge of the siRNA to the surface positive charge of the liposome were calculated to be 3:1 and 6:1, respectively.

In the following experiments, 10:7.6 and 5:1 were applied as the mixing ratio by weight of siRNA:DOTAP and the mixing ratio by weight of DOTAP:DOPE, respectively.

### TEST EXAMPLE 5

### Iontophoresis of Fluorescence-Labeled siRNA-Liposome Complex

10 μg of a Cy3-labeled siRNA (negative control siRNA purchased from Ambion, Inc.) were used and 200 μl of an siRNA-liposome complex suspension (mixing ratio by weight of siRNA:DOTAP was 10:7.6, mixing ratio by weight of DOTAP:DOPE was 5:1) were prepared in the same technique as in Test Example 4.

Next, according to the same technique as in Test Example 2, the Cy3-labeled siRNA-liposome complex suspension was filled in a cathode side of an electrode assembly of an iontophoresis device and 200 μl of a physiological saline solution were filled in an anode side of a device. Next, the iontophoresis device was mounted on a back skin of an SD rat shaved with clippers under Nembutal® anesthesia and iontophoresis was conducted at a constant current of 0.45 mA (0.15 mA/cm²) for 60 minutes, whereby the Cy3-labeled siRNA-liposome complex was administered.

### Preparation of Skin Slice and CLSM Observation

The skin was cut out immediately after the completion of iontophoresis, and the resultant was frozen and embedded in an OCT compound. A slice measuring 15 μm was prepared from the frozen skin sample with a cryostat and observed with an inverted confocal laser scanning microscope (LSM510 Carl Zeiss).

The results were as shown in FIG. 4. It was observed that the siRNA was permeated into a dermal layer by passing through a horny layer, epidermal layer, and basal membrane as the same as in the case where the siRNA-protamine complex was administered by iontophoresis. It was confirmed that, as the polycation substance, in addition to the protamine, a cationic liposome formed of the neutral lipid (DOPE) and the cationic lipid (DOTAP) can be used.

### TEST EXAMPLE 6

### Evaluation of Effect of Suppressing Gene Expression by siRNA-Liposome Complex

In order to evaluate the effect of suppressing a gene expression by intradermal delivery of an siRNA-liposome complex, experiments were conducted according to the following procedures.

Preparation of MAPK-1 siRNA-Liposome Complex

A MAPK-1 siRNA-liposome complex was prepared (mixing ratio by weight of siRNA:DOTAP was 10:7.6, mixing ratio by weight of DOTAP:DOPE was 5:1) using 5 μg (100 μl) of MAPK-1 siRNA (purchased from QIAGEN) in the same technique as in Test Example 4.

Iontophoresis of MAPK-1 siRNA-Liposome Complex and Quantitative Determination of MAPK-1 mRNA Level

A MAPK-1 siRNA-liposome complex was administered to a back skin of an SD rat by iontophoresis in the same conditions as in Test Example 3, whereby a MAPK-1 mRNA was determined quantitatively.

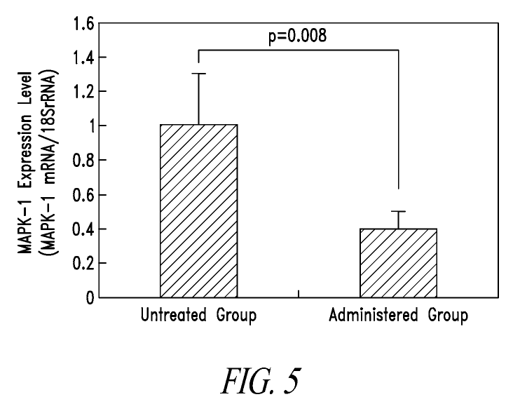

The results were as shown in FIG. 5. The MAPK-1 mRNA amount in the skin of the siRNA administration group decreased to about 39% of the mRNA amount of the untreated group. Further, the MAPK-1 mRNA amount of the siRNA-liposome complex administration group was significantly low compared to that of the untreated group (name of statistical test, Student's t-test).

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ structures and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

I/We claim:

1. A composition for iontophoresis, comprising:
an siRNA-polycation complex which is charged negatively.

2. The composition according to claim 1 wherein the siRNA-polycation complex comprises an siRNA and a polycation which are bound to each other by an electrostatic interaction.

3. The composition according to claim 1 wherein the siRNA-polycation complex has a zeta potential of from −50 to −5 mV.

4. The composition according to claim 1 wherein a negative to positive (−/+) charge ratio of the siRNA to the polycation is from 2:1 to 7:3.

5. The composition according to claim 1 wherein the siRNA-polycation complex has an average particle diameter of from 50 to 3,000 nm.

6. The composition according to claim 1 wherein the siRNA has a total number of negative charges of from 30 to 60.

7. The composition according to claim 1 wherein a sense strand and an antisense strand of the siRNA each have from 15 to 30 nucleotides.

8. The composition according to claim 1 wherein a double-stranded part of the siRNA has from 15 bp to 28 bp.

9. The composition according to claim 1 wherein the polycation is a cationic protein or a cationic liposome.

10. The composition for iontophoresis according to claim 9 wherein the cationic protein has a total number of positive charges of from 1 to 100.

11. The composition according to claim 9 wherein the cationic protein has a molecular weight of from 500 kDa to 50,000 kDa.

12. The composition according to claim 9 wherein the cationic protein is at least one selected from the group consisting of protamine, poly-L-lysine, an arginine oligomer, and a lysine oligomer.

13. The composition according to claim 9 wherein the cationic liposome has an average particle diameter of from 50 to 1,000 nm.

14. The composition according to claim 9 wherein the cationic liposome includes at least a cationic lipid.

15. The composition according to claim 14 wherein the cationic lipid is a C12-20 lipid having a positive charge of from 1 to 10 valences.

16. The composition according to claim 14 wherein the cationic lipid is selected from the group consisting of 1,2-dioleoyloxy-3-(trimethylammonium)propane, dioctadecyl dimethyl ammonium chloride, N-(2,3-dioleoyloxy)propyl-N,N,N-trimethyl ammonium, didodecylammonium bromide, 1,2-dimyristoyloxypropyl 1,3-dimethylhydroxyethyl ammonium, and 2,3-dioleoyloxy-N-[2(spermine carboxyamide)ethyl]-N,N-dimethyl-1 -propanamium trifluoroacetate.

17. The composition according to claim 16 wherein the cationic lipid is 1,2-dioleoyloxy-3-(trimethylammonium)propane.

18. The composition according to claim 14 wherein the cationic liposome further comprises a neutral lipid.

19. The composition according to claim 18 wherein the neutral lipid is selected from the group consisting of diacylphosphatidyl ethanol amine and diacylphosphatidyl choline.

20. The composition according to claim 19 wherein the neutral lipid is selected from the group consisting of dioleylphosphatidyl ethanol amine.

21. The composition according to claim 1 which is in a dry form.

22. The composition according to claim 1 which is used as a drug.

23. An electrode assembly for iontophoresis, comprising:
an electrode; and
an siRNA complex holding portion holding a composition comprising an siRNA-polycation complex which is charged negatively, the holding portion being provided in a skin side of the electrode, wherein
the electrode assembly is capable of intradermally administering to an organism the siRNA-polycation complex by iontophoresis.

24. The iontophoresis device according to claim 23, further comprising:
a counter electrode assembly; and
an electric power unit,
the electrode assembly electrically coupled to a cathode of the electric power unit, and
the counter electrode assembly electrically coupled to an anode of the electric power unit.


SEQUENCE LISTING (5 sequences)

SEQ ID NO: 1 - DNA, 21 bp, Artificial Sequence
  One strand of siRNA
cccucacaagaggauugaatt

SEQ ID NO: 2 - DNA, 21 bp, Artificial Sequence
  One strand of siRNA
uucaauccucuugugagggtt

SEQ ID NO: 3 - protein, 32 aa, Homo sapiens
PRRRRSSSRPVRRRRRPRVSRRRRRRGGRRRR

SEQ ID NO: 4 - DNA, 21 bp, Artificial Sequence
  Primer
agttgaacaggctctggccca

SEQ ID NO: 5 - DNA, 21 bp, Artificial Sequence
  Primer
tgaacgcgtccagtcctctga